(19) United States
(12) Patent Application Publication
(10) Pub. No.: US 2017/0368287 A1
(43) Pub. Date: Dec. 28, 2017

(54) Molded vinyl/polyvinyl/rubber/silicone oxygen mask contoured for improved compfort of user with acute or chronic need for oxygen or aerosolized medications. Mask applicable for all adult and pediatric oxygen masks. New decreased size of mask with gel strip at nasal bridge and under-the-chin contiguous portion of molded mask body modifications.

(71) Applicant: Michael Edward Labrecque, Los Lunas, NM (US)

(72) Inventor: Michael Edward Labrecque, Los Lunas, NM (US)

(21) Appl. No.: 14/999,569

(22) Filed: May 26, 2016

Related U.S. Application Data
(60) Provisional application No. 62/236,012, filed on Oct. 1, 2015.

(51) Int. Cl. A61M 16/06; A61M 16/00; A61M 16/14; A61M 11/00; A61M 16/08; A61M 16/20

(52) U.S. Cl. CPC A61M 16/0616; A61M 16/0875; A61M 16/208; A61M 16/14; A61M 11/00; A61M 16/0683; A61M 16/0078; A61M 2202/0208

(57) ABSTRACT

An oxygen face mask composed of a molded vinyl/polyvinyl/rubber or silicone mask with a medical grade mineral oil gel strip on the bridge of the nose extending over the contour of the cheeks on both side of the patient's face, an extended under-the-chin portion of mask and decreased length dimension of an existing standard face mask to deliver oxygen and aerosolized medications to the user. Use of this face mask during aerosolized medication of medical gas or oxygen delivery allows for a better seal against the face, greater comfort to the user, more stability and good securement of the mask, and a mask size that decreases irritation of the user's eyes caused by leakage at the nose and cheek portions of a standard oxygen mask. The oxygen mask configuration can applied to both pediatric and adult patients.

### BACKGROUND OF THE INVENTION

The present invention relates to oxygen masks and, more particularly, to an oxygen face mask with a gel nasal strip, an under-the-chin segment of the mask and a reduced dimension of the overall mask for use in patients requiring aerosol medication delivery and oxygen therapy for both pediatric and adult patients.

Oxygen and aerosolized delivery by mask allows for medication and medical gases to be delivered to a patient who requires supplemental oxygen or aerosolized medications. This method of oxygen/aerosolized medication delivery may be inadequate for patients with compromised pulmonary function, COPD, Asthma, respiratory failure, either acute or chronic, or hypoxic patients because medication and oxygen delivery is not maximized due to leakage around the nose and eye portion of a standard mask in part to its overall size and rolled edge at the nasal portion of the mask. The use of a metal strip that does not adequately seal the nose and cheek area is subjectively applied pressure to the metal strip that can cause discomfort to the nose and upper cheek areas. The size of a standard oxygen face mask may prohibit the use of eyewear by the patient which interferes with the user's daily activities of living. As can be seen, there is a need for a greater deposition of oxygen and aerosolized medications that is enhanced by the gel strip fit against the face, elimination of the rolled edge at the nasal portion of the mask and elimination of the metal adjustment strip on the nose which will allow for greater comfort and function of the mask. The additional under-the-chin segment of the mask body allows for a greater stability of the mask in the case of movement of the face or head by the user during use of the mask. The reduced size of the mask at the nose portion allows for the utilization of eyewear during therapy.

### SUMMARY OF THE INVENTION

In one aspect of the present invention, an oxygen mask comprises a mask adapted to fit over a nose, mouth and chin of a patient, pediatric or adult, a nasal gel strip in a portion of the mask and an under-the-chin portion of the mask. The mask having a reduced size for a more comfortable fit, more enhanced securement of the mask and an improved seal of delivered oxygen or aerosolized medication to the patient, pediatric and adult, are aspects and advantages that will be understood with reference to the following drawings, descriptions and claims.

### BRIEF DESCRIPTION OF THE DRAWINGS

FIG. 1 is a detailed side view of an oxygen face mask showing a gel nasal strip in connection with the mask and an under-the chin extended portion of the mask, in use, according to an exemplary embodiment of the present invention.

FIG. 2 is a detailed side view of an oxygen mask and a user's face showing the location of a gel nasal strip in connection with the reduced size of the mask on the face, a space between the top of the mask and the forehead to allow for the use of eyewear, the mask is void of a metal adjustment strip at the nose and comprising an extended under-the-chin portion of the mask, in use, according to an exemplary embodiment of the present invention.

### DETAILED DESCRIPTION OF THE INVENTION

The following detailed description is the best currently contemplated modes of carrying out the exemplary embodiments of the invention. This detailed description is not intended to describe each embodiment or every implication. The figures and the detailed description, as follows, more particularly exemplify illustrative embodiments.

Broadly, an embodiment of the present invention provides an oxygen face mask with a gel nasal strip that provides a proper, comfortable seal of the mask and an under-the-chin portion of the mask body that provides increased securement from movement on the patient's face. Use of the face mask with the nasal strip, under-the chin portion of the mask and the reduction in size provides for increased delivery of oxygen and or aerosolized medications. The reduction in size and the lack of a metal nasal strip allows the patient to utilize eyewear without physical restrictions from the mask.

Referring now to:

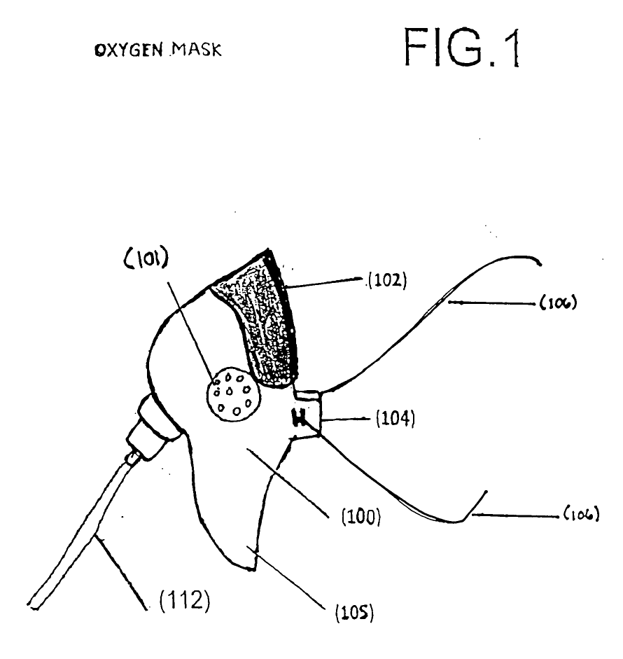

FIG. 1 is the oxygen face mask 100 secured to the face by one or two elastic cords 106 attached to slits 104 on the sides of the mask. The gel nasal strip 102 covers the nose and creates a seal with the nose, cheeks and the mask. An under-the-chin portion of the mask 105 provides addition securement of the mask to the patient's face. Side ports 101 allow for elimination of CO2 from the mask. Oxygen is supplied by tubing 112 from an oxygen source. A fixed connector 107 on the mask attaches the oxygen delivery tubing to the mask.

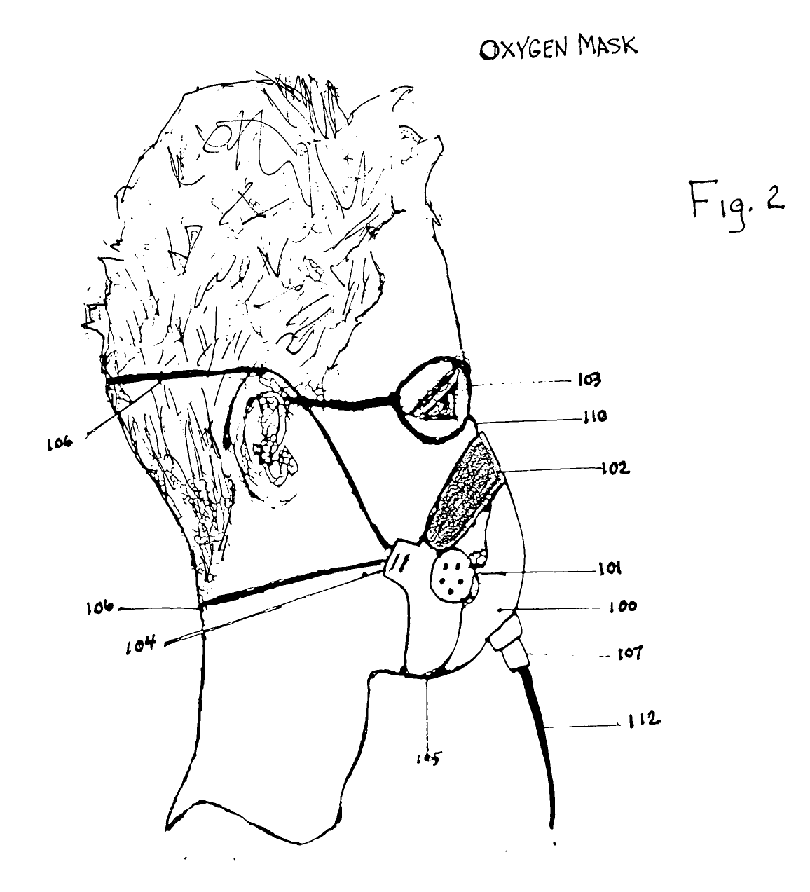

FIG. 2 is the oxygen face mask 100 secured to the face by two elastic cords 106 attached to slits 104 on the sides of the mask. The gel nasal strip 102 covers the nose and creates a seal with the nose, cheeks and mask. An under-the-chin portion of the mask 105 provides additional securement of the mask to the patient's face. Side ports 101 allow for elimination of CO2 from the mask. Oxygen is supplied by tubing 112 from an oxygen source. A fixed connector 107 on the mask attaches the oxygen delivery tubing to the mask. The facial side perspective shows eyewear 103 on the user's eyes and the space 110 between the reduced size mask nose portion and the forehead which allows for eyewear to be worn by user.

It should be understood, of course, that the foregoing relates to exemplary embodiments of the invention and that modifications may be made without departing from the spirit and the scope of the invention as set forth in the claims.

### REFERENCES CITED

### U.S. Patent Documents

| | | | |
|---|---|---|---|
| 6,418,929 | Jul. 8, 1941 | Lombard, Charles | Method and apparatus |
| | | | for administering oxygen |
| US2843121A | May 20, 1955 | Hudson, Charles | Oxygen Mask |
| U.S. Pat. No. 4,167,185 | Sep. 11, 1979 | Lewis, Robert | Face Mask Seal |
| 2,269,461 | Jan. 13, 1942 | Lehmberg, William | Respirator |
| 6,112,746 | Sep. 5, 2000 | Kwok, Phillip | Nasal Mask and Mask |
| | | | Cushion Therefor |
| 5,921,239 | Jul. 13, 1999 | McCall, et al. | Face mask for patient |
| | | | breathing |
| 6,418,929 | Jul. 16, 2002 | Norfleet, Suzanne | Infasnt Oxygen Mask |
| US 2012/0285448 A1 | Nov. 15, 2012 | Dugan, et al | Tube placement in non-invasive |
| | | | ventilation |
| US 2013/0060157 A1 | Mar. 7, 2013 | Beard,John | Oxygen mask with Capnography |
| | | | Monitoring Ports |

What is claimed is:

1-13. (canceled)

14. An oxygen face mask adapted to conform and fit over a nose, mouth and under the chin of a user that is attached to an oxygen source that delivers oxygen to the user.

15. The oxygen face mask of claim 1, to include a gel strip adhered on the inside portion of the oxygen face mask, to cover vertically, at the least, ⅓ of the distance from the tip of the nose to the junction of the nose with forehead and laterally at the least ⅓ the distance from the midpoint of the top surface of the nose, across the cheeks and to the edge of the jaw line.

16. The oxygen face mask of claim 2 relates to a face, at least, with a nose 60 mm long and 110 mm from the midpoint of the top surface of the nose to the jaw line.

17. The oxygen face mask of claim 2 further comprising a medical grade mineral oil gel strip, with adhesive qualities, not less than 5 mm in width, 10 mm 34 in length and 2 mm in thickness with semi-circular ends.

18. The oxygen face mask of claim 1, further comprising an under-the chin stabilizing segment of the mask incorporated into the face mask at least 40 mm from the midpoint of the base of the chin to the throat.

19. The oxygen face mask of claim 1, further comprising one or two elastic straps attached to each side of the face mask through strap holes in the mask and operable to provide support and securement of the mask to the user's face.

20. The oxygen face mask of claim 1 is void of any metal nasal adjustment strip used to manually adjust the fit of the mask to the user's face.

21. The oxygen face mask of claim 1, further comprises a mask void of a rolled edge at the nose portion of the user's face

22. The oxygen face mask of claim 1 is reduced to the oxygen face mask in claim 1, comprising a mask with a dimensions not greater than, 140 mm in length from the top of the mask to the base of the chin and not greater than 100 mm at its widest point.
